(19) United States
(12) Patent Application Publication
(10) Pub. No.: US 2006/0078875 A1
(43) Pub. Date: Apr. 13, 2006

(54) Genetic selection of small molecule modulators of protein-protein interactions

(75) Inventors: Stephen J. Benkovic, State College, PA (US); Alexander R. Horswill, Coralville, IA (US); Sergey Savinov, West Lafayette, IN (US)

Correspondence Address: WELSH & KATZ, LTD, 120 S RIVERSIDE PLAZA, 22ND FLOOR, CHICAGO, IL, 60606, US

(73) Assignee: The Penn State Research Foundation

(21) Appl. No.: 11/232,339

(22) Filed: Sep. 21, 2005

Related U.S. Application Data
(60) Provisional application No. 60/612,337, filed on Sep. 23, 2004.

(51) Int. Cl. C12Q 1/00; C12Q 1/68; C12N 1/18; C12N 5/06; C12N 1/21

(52) U.S. Cl. 435/4; 435/6; 435/252.3; 435/254.2; 435/325

(57) ABSTRACT

The present invention provides a method of production and screening of small molecule modulation of inter-macromolecule interaction. The method involves providing a living cell containing a gene that directs expression of a gene product to be assayed for the ability to modulate inter-macromolecule interactions and an inter-macromolecule interaction whose interaction can be monitored. The inter-macromolecule interaction is monitored in the living cell to determine if the inter-macromolecule interaction is modulated in the living cell relative to another, otherwise similar living cell that lacks said gene product.

### RELATIONSHIP TO OTHER APPLICATIONS

This application claims the benefit under 35 U.S.C. § 119(e) of prior U.S. Provisional Patent Application 60/612,337 filed Sep. 23, 2004.

### GOVERNMENTAL SUPPORT

The present invention was made with governmental support pursuant to USPHS grant GM 24129 DE13964 and DE13088 from the National Institutes of Health. The government has certain rights in the invention.

### TECHNICAL FIELD

This invention relates to the fields of high-throughput pharmaceutical identification and screening, in vivo genetic screening, and of protein biology, and more particularly to the use of transformed cells to perform in vivo screening of in vivo produced modulators of inter-macromolecule interactions.

### BACKGROUND ART

Many regulatory processes in living organisms are a consequence of specific protein-protein contacts, and interference with such interactions provides a means to control specific cellular events. The de novo discovery of small molecules capable of disrupting such protein-protein complexes has been fraught with challenges, yielding very few inhibitors at a low success rate [Cochran (2000) Chem. Biol. 7:R85-94.; Toogood (2002) J. Med. Chem. 45:1543-1558.; Berg (2003). Angew. Chem. Int. Ed. 42:2462-2481]. These difficulties suggest that vast libraries with high functional diversity might be essential for finding unusual molecules that are capable of perturbing the intracellular levels of protein-protein complexes. The major challenge in sifting through such large compound pools is the availability of functional high-throughput assays for detection of the protein complex association and dissociation.

Genetic selection is uniquely able to rapidly identify individual molecules with the desired properties from large libraries. The application of this concept involves whole cells acting as reporters, which correlates host growth to a desired functional property. Unlike recently popularized affinity selections [Lin et al. (2002) Angew. Chem. Int. Ed. Engl. 41:4402-4425], an intracellular genetic selection can directly assay for effects on enzymatic activity or the modulation of a protein-protein complex, thus bypassing inherent limitations of in vitro approaches [Taylor et al. (2001) Angew. Chem. Int. Ed. Engl. 40:3310-3335].

Additionally, because library members must function within the context of the entire host proteome, positive candidates have an enhanced level of selectivity for their target. This represents an important advantage over traditional screen-based methods in pharmaceutical discovery and development by permitting both target affinity and selectivity to be simultaneously optimized. If genetic selection could be applied to the discovery of small molecule modulators of cellular regulatory processes, then throughput of assays would be greatly enhanced to potentially yield both potent and selective activities as well as novel modes of action.

High-throughput genetic selections have shown remarkable promise in yielding rare candidates with desired properties. The ability to monitor small-molecule mediated association (FKBP12-Rapamycin-FRAP and others) and dissociation (HIV-1 protease, mammalian ribonucleotide reductase and others) of protein complexes provides a potent system for genetic selections against libraries of protein effectors and in principle permits the full range of effects on the monitored interaction, including e.g., stabilization or inhibition of interactions by the effector.

The present invention addresses these problems by utilization of a method for producing and screening libraries of in vivo produced candidate modulators of inter-macromolecule interactions.

### BRIEF SUMMARY OF THE INVENTION

One aspect of this invention contemplates a method for in vivo production and screening of modulators of inter-macromolecule interactions. In this method, a living cell is provided that contains (i) a gene that directs expression of a gene product to be assayed for the ability to modulate inter-macromolecule interactions such as protein-protein interactions and (ii) inter-macromolecule interactions such as protein-protein interaction whose interaction can be monitored. The inter-macromolecule interactions are monitored in the living cell; and whether the inter-macromolecule interaction is modulated in the living cell relative to another, otherwise similar living cell that lacks said gene product is determined.

Another aspect of this invention is a living cell in which genes can be assayed for their ability to modulate an inter-macromolecule interaction that can be monitored in vivo. The living cell can be a bacterial cell, but in some embodiments of the invention the living cell is eukaryotic.

In some aspects of this invention, the gene to be assayed comprises a library of genes, in which case the library components are introduced into a plurality of living cells such that a plurality of library components are simultaneously assayed for their ability to modulate an inter-macromolecule interaction in vivo.

The gene to be assayed can encode a small molecule such as a peptide that is an effector or modulator of an inter-macromolecule interaction. The gene to be assayed can encode a library of peptides, such as a SICLOPPS library [Abel-Santos et al. (2003) Methods Mol. Biol. 205:281-294]. The gene to be assayed can, alternatively, encode an enzyme or group of enzymes that catalyze the formation of an active molecule such as a macrolide or steroid that modulates the inter-macromolecule interaction or otherwise results in the indirect modulation of the interaction. The gene to be assayed, again in the alternative, can encode a nucleic acid that modulates the monitored interaction.

A particular aspect of this invention is a method for in vivo production and screening of small molecule modulation of an inter-macromolecule interaction. This method includes the steps of providing a living cell having an inter-macromolecule interaction that can be monitored in vivo, providing a gene that directs expression of a small molecule, e.g., peptide, gene product to be assayed for the ability to modulate the inter-macromolecule interaction, and monitoring the interaction in vivo to determine if it is thereby modulated, where the gene product is or causes the production of the small molecule.

Another aspect of this invention is a method for screening for promoters as well as inhibitors of inter-macromolecule interactions.

### BRIEF DESCRIPTION OF THE DRAWINGS

In the drawings forming part of this invention,

FIG. 1, in three parts, A-C, is a schematic map of reverse two-hybrid system (RTHS) plasmids for making repressor fusions: A, Plasmid pTHCP14 for constructing heterodimeric fusions for strains (SNS126 derivatives) containing the chimeric operator. B and C, Plasmid pTHCP16 and plasmid pTHCP17, respectively, for constructing fusions for strains (SNS118 derivatives) containing phage 434 operator sequences.

FIG. 2 shows the sequence of the promoter regions used. 2A, Promoter region with chimeric 434·P22 operator sequences. The sequence of the anti-sense (bottom) strand, including the 5′ XbaI and 3′ PstI site overhang is

5′ CTAGAT ATTTAAGAT TTCTTGT ATTTTC ATTTAAGAT ATCTTGT T TGTCAA AT CTGCA (SEQ ID:01).

FIG. 2B, Promoter with wild-type 434 operator. The sequence of the anti-sense (bottom) strand, including the 5′ XbaI and 3′ PstI site overhang is

5′ CTAGAT ACAAGAT TTCTTGT ATTTTC ACAAGAT ATCTTGT T TGTCAA AT CTGCA (SEQ ID:02).

FIG. 3 is a graph illustrating β-galactosidase assays for testing strain selectivity with the FKBP12-FRAP pairing. Fusions with either phage 434 wild-type or 434·P22 chimeric DNA-binding domains were integrated into strains containing either 434 wild-type (SNS118) or 434·P22 chimeric (SNS126) promoters. β-Galactosidase assays were performed without (white bars) and with 10 μM rapamycin (black bars) for each strain type.

FIG. 4 has two graphs (A and B) of β-galactosidase assays showing the effect of linear peptide inhibitors on the oligomeric state of HIV-1 protease and ribonucleotide reductase. FIG. 4A shows a comparison of the effect of the known inhibitor (pHIV16) versus the scrambled control (pHIV17) within strain SNS118 expressing an integrated HIV-1 protease fusion at arabinose concentrations of 0, 33, and 66 μM. FIG. 4B shows a comparison of the effect of the known inhibitor (pTHCP35) versus the scrambled control (pTHCP37) within strain SNS126 expressing an integrated ribonucleotide reductase fusion at IPTG concentrations of 0, 10, and 30 μM.

FIG. 5 contains two graphs, 5A and 5B, that illustrate optimization of 3-AT and kanamycin concentrations, respectively, for genetic selections of ribonucleotide reductase dissociative inhibitors. Biomass of a culture of strain SNS126 with an integrated ribonucleotide reductase fusion was grown with increasing 3-AT (FIG. 5A) and kanamycin (Fig. with increasing 3-AT (FIG. 5A) and kanamycin (FIG. 5B) concentration and normalized against a culture of null integrant grown under the same conditions.

FIG. 6 in two parts (A and B) shows schematic representations of RTHS. FIG. 6A illustrates the expression of protein fusions containing DNA-binding domains induced with IPTG, and associate to repress a promoter that directs expression of three reporter genes: HIS3 (imidazole glycerol phosphate dehydratase; IGPD); Kan^{R}, (aminoglycoside 3′-phosphotransferase); lacZ, (β-galactosidase.) The stippled rectangles represent DNA-binding protein domains fused to interacting proteins (hatched shapes). The formation of protein complexes inhibits growth on minimal media by blocking HIS3 expression, and residual background expression is chemically tunable with 3-AT (competitive inhibitor of IGPD) and kanamycin. The final reporter, β-galactosidase, quantitatively reports on the level of repression. In FIG. 6A, a heterodimer interaction inhibits expression of the downstream genes, but the repressor complex can form from a single fusion protein type when an interacting protein domain (hatched shape) can form a homodimer. FIG. 6B illustrates that a small-molecule modulator (diamond shape) capable of inhibiting the protein-protein interaction rescues growth by inducing HIS3 and Kan^{R} expression. When one of the proteins interacts instead with the small-molecule modulator, the repression complex of 6A is not formed.

FIG. 7, in four parts (A-D), illustrates processing of ribonucleotide reductase candidates. FIG. 7A shows sequences of variable inserts, listed in order of biological activity. These are:

| | | |
|---|---|---|
| pRR-112 | VKFWF | (SEQ ID: 03) |
| pRR-130 | RYYNV | (SEQ ID: 04) |
| pRR-93 | YTWSY | (SEQ ID: 05) |
| pRR-58 | IPLLY | (SEQ ID: 06) |
| pRR-127 | GVRFF | (SEQ ID: 07) |
| pRR-184 | LNYLW | (SEQ ID: 08) |
| pRR-133 | HRYVF | (SEQ ID: 09) |
| pRR-131 | KISLF | (SEQ ID: 10) |
| pRR-120 | VLYSW | (SEQ ID: 11) |

FIG. 7B shows a bar graph of β-galactosidase assays that illustrate the in vivo potency of four expressed peptides as a function of the arabinose concentration. Positive (unrepressed strain) and negative (SICLOPPS control plasmid) controls are provided as reference points. Assays were performed at 100 μM IPTG to induce ribonucleotide reductase expression, and the inset graph shows a titration that identified this optimal level of IPTG. FIG. 7C is a graph of competition ELISA results that compare the binding affinity of the four linear peptides with P8 control. Relative IC₅₀ values are listed in Table 1. FIG. 7D illustrates an exemplary solid phase synthesis of cyclic peptides. First, an activated disulfide resin is prepared through the protection of the thiol group of 3-mercaptopropionic acid, followed by coupling to an amino-PEGA resin. Next, a linear peptide is attached via a cysteine residue and cyclized with 1-ethyl-3-(3′-dimethylaminopropyl)carbodiimide (EDC) and 1-hydroxy-7-azabenzotriazole (HOAt) in DMF. Finally, reductive cleavage with tris-2-carboxyethylphosphine (TCEP) releases the cyclized peptide.

TABLE 1
Relative inhibition of ribonucleotide reductase
protein-protein interaction by linear peptides

| Peptide | Relative IC₅₀ |
|---|---|
| 1-RR-127 | 9.8 |
| 1-RR-130 | 4.0 |
| 1-RR-133 | 2.8 |
| 1-RR-93 | 2.0 |
| P8 | 1.0 |

FIG. 8A-C shows results from an immobilized peptide ELISA. FIG. 8A shows an assay schematic showing immobilized peptide (small open rounded rectangle) being recognized by a protein receptor (shaded larger rounded rectangle; e.g., mR1 or mR2), which in turn is being detected via a His6 tag by Ni·NTA-HRP conjugate (Qiagen; HRP, horseradish peroxidase; shaded circle=Ni, covalently bound Ni cation). FIG. 8B provides the results of an immobilized P8 ELISA. Data demonstrate specific recognition of P8 control peptide by ribonucleotide reductase large subunit (mR1), which was verified (not shown) by measuring disruption of P8·mR1 complex due to incubation with peptides l-RR93 and c-RR93 versus P8 as a reference peptide. FIG. 8C shows immobilized c-RR130 ELISA. Data demonstrate specific recognition of c-RR130 control peptide by ribonucleotide reductase small subunit (mR2), which was similarly verified (not shown) by measuring disruption of c-RR130·mR2 complex due to incubation with peptides l-RR127, c-RR127, l-RR130, and l-RR133.

FIG. 9 illustrates the final two steps of the de novo purine biosythesis pathway catalyzed by ATIC

FIG. 10 is a schematic depiction of how a expressed fusion protein can fold to form an active Intein, which undergoes a series of rearrangements to generate a cyclic peptide. In this case the target cyclic peptide contains a series of randomly encoded amino acids forming libraries of about 10⁸ members. Library 1: Z=S, Target Peptide=CX₁X₂X₃X₄X₅ (SEQ ID:12); Library 2: Z=O, Target Peptide=SGWX₁X₂X₃X₄X₅ (Xₙ=random amino acid) (SEQ ID:13).

FIG. 12 is a graph showing the Kᵢ of cyclic peptide inhibitors 1a and 151 and their linear counterparts, determined by assuming competitive inhibition with respect to 10-f-THF.

FIG. 13 gives the wild type 434 promotor structure of the plasmids used in Examples 7-9. The boxed regions, O₁1₄₃₄ and O₂1₄₃₄, are the binding sites for the repressor domains. Underlined sequences are the −35 and −10 transcription signals, as indicated. The sequence of the anti-sense (bottom) strand, including the 5′ and 3′ overhangs is 5′ CTAGA TCA ACAAAACTTTCTTGT ATTTTC AT ACAATGTATCTTGT T TGTCAA AT CTGCA 3′ (SEQ ID:14)

The present invention has several benefits and advantages. One advantage of the present invention is that the power of positive genetic selection can be applied to high-throughput drug screening, permitting extremely rare, effective individuals to be selected from an extremely large library of potential effectors.

A benefit of the present invention is that novel modes of action can be found because genetic screens are not biased toward any specific mode of action, e.g., where a protein-protein interaction is monitored, effectors can be identified that bind to each of the proteins, rather than just one as with in vitro affinity-based screens.

Another advantage of the invention is that interaction modulation is observed in an in vivo environment, including the entire proteome of the living cell, so increased selectivity can be had relative to in vitro assays, which occur in abiotic conditions.

Another benefit of the present invention is that the entire range of gene expression products, from RNA to peptides to secondary metabolites can be assayed for modulating effect.

Yet another advantage of the present invention is that sensitivity of the living cell to interaction modulation, which can be related to specific affinity and selectivity of the effector, can be adjusted.

Yet another benefit of the present invention is that the entire range of possible modulation of interactions, from promotion and stabilization of interaction to inhibition of interaction, can be examined.

A further advantage is the ability to have synergistic reporter effects, in that the same interaction can be monitored using a plurality of genetic reporter systems within the same cell, further improving sensitivity, selectivity, and adaptability of the method.

A further benefit of the present invention is that the process is adapted to high-throughput in vivo analyses of a large number of effector candidates.

Another advantage is that bacterial or eukaryotic cells can be used, as required by the experimental needs of the users.

### DETAILED DESCRIPTION OF PREFERRED EMBODIMENTS

A system for in vivo production and assaying of modulators of inter-macromolecule interactions is disclosed herein. This system builds on one-hybrid, reverse two-hybrid, and three-hybrid systems by incorporating in vivo production of a candidate modulator of macromolecule interaction, or effector, to be tested. In this system host cell survival and/or reporter gene expression is tied to the interaction of particular macromolecules in vivo and allows the interaction to be monitored. The ability of the candidate effector to promote or inhibit the particular interaction is thereby monitored by its correlation with cell survival or reporter gene expression. The system relies on conditional expression of two chromosomal reporters, enabling sensitive, chemically tunable genetic selections. This system provides a new technique for seeking new expressible pharmaceutical products and products derived from such expressible materials such as cyclic peptides and secondary metabolites.

The cell used in an in vivo method herein can be a prokaryote or a eukaryote. Substantially any culturable prokaryote can be used although a bacterium such as E. coli is preferred. Similarly, substantially any culturable eukaryote can be used such as yeast cells like those of Saccharomyces cerevisiae, animal cells such as those of a cancer or hybridoma, or plant cells such as algae, tobacco, or protoplasts thereof.

A contemplated gene product can be a nucleic acid (e.g. RNA), a peptide, a steroid or a macrolide. An exemplary peptide can have a length of about 4 to about 150 or more residues. Preferably, the peptide has a length of about 5 to about 50 residues. Steroids and macrolides are well-known secondary products of expressed genes and rapamycin is illustrative of the group.

As used herein, “small molecules” includes peptides up to about 150 residues in length, nucleic acids up to about 150 bases and secondary metabolites such as steroids and macrolides that are products of enzyme action in vivo. Such small molecules and analogues thereof can be synthesized in vitro by known techniques for continued analysis and characterization.

One aspect of this invention is a method for in vivo production and screening of modulators of inter-macromolecule interactions. This method includes the steps of providing a living cell having an inter-macromolecule interaction that can be monitored in vivo, and a gene directing expression of a gene product to be assayed for the ability to modulate the inter-macromolecule interaction. The in vivo inter-macromolecule interaction is monitored to determine if the interaction is thereby modulated. The inter-macromolecule interaction can be a protein-protein interaction or a protein-nucleic acid interaction or a combination thereof.

Another aspect of this invention is a living cell in which genes can be tested for their ability to modulate an inter-macromolecule interaction that can be monitored in vivo. The living cell can be a bacterial cell, but in some embodiments of the invention the living cell will be eukaryotic.

In some versions of this invention, the gene to be tested comprises a library of genes, in which case the library components are introduced into a plurality of living cells such that a plurality of library components are simultaneously tested for their ability to modulate an inter-macromolecule interaction in vivo.

The gene to be tested can encode a peptide that is a potential effector of an inter-macromolecule interaction. The gene to be tested can be a library encoding a library of peptides, such as a SICLOPPS library. The gene to be tested can, alternatively, comprise an enzyme that catalyzes the formation of an active molecule that potentially modulates the inter-macromolecule interaction or otherwise results in the indirect modulation of the interaction. The gene to be tested, again in the alternative, can encode a nucleic acid that modulates the monitored interaction.

Another aspect of this invention is a method for in vivo production and screening of small molecule modulation of an inter-macromolecule interaction. This method includes the steps of providing a living cell having an inter-macromolecule interaction that can be monitored in vivo, and a gene directing expression of a small molecule gene product such as a peptide to be tested for the ability to modulate the inter-macromolecule interaction, and monitoring the interaction in vivo to determine if it is thereby modulated, where the gene product is or directs the production of the small molecule.

Another aspect of this invention is a method for screening for promoters as well as inhibitors of inter-macromolecule interactions.

Thus, a bacterial cell capable of identifying small molecule modulators of inter-macromolecule, including protein-protein, interactions is illustrated herein. The SICLOPPS technology is ideally suited to interface with this system, and the compartmentalization of both methodologies within cells permits the discovery of cyclic peptide disruptors through genetic selection. By challenging each candidate against the host proteome, without eliciting toxic effects, the selected peptides can display a degree of target selectivity, a critical concern for drug development. The implementation of this illustrative approach toward ribonucleotide reductase identified four peptides that disrupted the enzymatic complex by two different mechanisms. The chemical cyclization of these peptides, using a novel solid phase scheme, improved their relative binding affinity.

Although the activities found within the hexapeptide library were comparable to the existing linear inhibitor, the selected epitopes are now presented from pharmacologically tractable, structurally better defined scaffolds, amenable to further optimization. Towards this goal, the chemical composition displayed by selectants can be grafted onto peptidomimetic platforms with improved pharmacokinetic and structural properties [Hirschmann et al. (1998) J. Med. Chem. 41:1382-1391]. Additionally, the unprecedented binding modes of some peptides highlighted the advantages of using a genetic selection. Considering the key nature of inter-macromolecule, including protein-protein, interactions for many physiological functions and the unique properties of these interfaces, the ability to systematically identify modulators of these interactions can open new avenues in drug development.

### EXAMPLE 1

### Proteins, Peptides and Interaction Analyses

A bacterial reverse two-hybrid system and a three-hybrid system are described that are capable of correlating host cell survival and/or reporter gene expression to the interaction of proteins in vivo. The system relies on conditional expression of two chromosomal reporters, enabling sensitive, chemically tunable genetic selections.

By subjecting the ribonucleotide reductase complex to a SICLOPPS library, cyclic-peptide dissociative inhibitors were identified that yielded several potent effectors, some with an unexpected binding mode, highlighting the intrinsic strength of genetic selection. Given the large library population that a bacterial selection system can potentially process, this method could become a powerful tool for identifying uniquely active modulators of protein-protein interactions.

Cyclic Peptide Synthesis

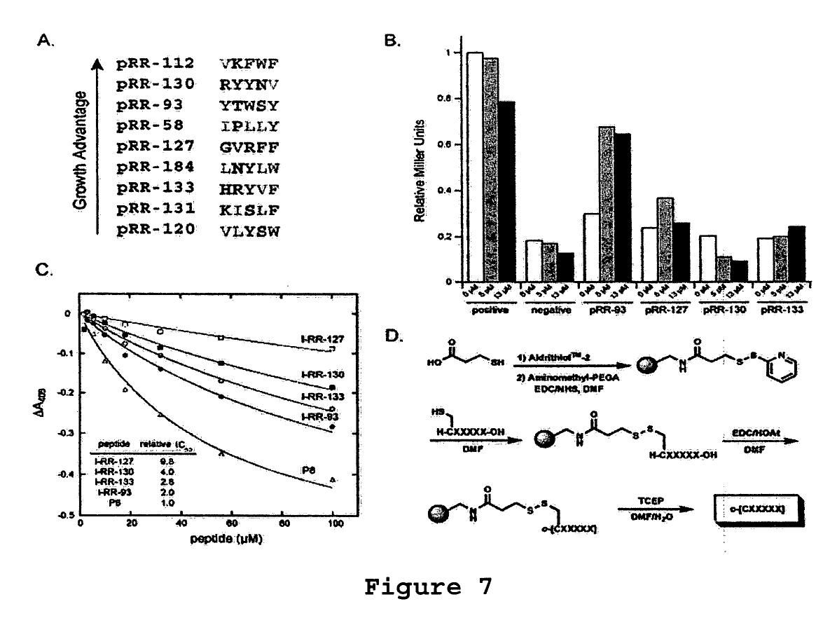

3-Mercaptopropionic acid (69 mg, 0.65 mmol) was reacted with 2-Aldrithiol™ (Aldrich, 179 mg, 0.81 mmol) in 500 μL of N,N-dimethylformamide (DMF), and the completion of the reaction was monitored by the release of 2-thiopyridone (λₘₐₓ 353 nm, ε=8080 M⁻¹cm⁻¹). The reaction product was then coupled in situ with amino PEGA resin (Novabiochem, ca. 0.325 mmol) using 1-ethyl-3-(3′-dimethylaminopropyl)-carbodiimide (EDC, 125 mg, 0.65 mmol), N-hydroxysuccinimide (HOSu, 112 mg, 0.98 mmol), and N,N-disopropylethylamine (210 mg, 1.63 mmol). Loading of the resulting disulfide resin (ca. 0.23 mmol/g) was established by displacing 2-thiopyridone with large excess of cysteine. An aliquot of the resin (0.006 mmol) was incubated with cysteine containing peptides (0.012 mmol) in 500 uL of DMF, and the progress of the peptide attachment was again monitored spectrophotometrically. Immobilized peptide was cyclized with EDC (3.5 mg, 0.018 mmol) and 1-hydroxy-7-azabenzotriazole (HOAt, 4.9 mg, 0.036 mmol) in 650 μL of DMF, and the progress of the cyclization was monitored by Kaiser assay [Kaiser et al. (1970) Anal. Biochem. 34:595-598]. Finally, reductive cleavage with tris-2-carboxyethylphosphine (TCEP, 17.2 mg, 0.06 mmol) in 50% aqueous DMF (1 mL) released cyclic peptides from the resin. Crude peptide mixtures were subjected to reverse-phase (C18 Partisil M9 10/50 ODS-3; Whatman) chromatography on Waters HPLC system using water/acetonitrile gradient with 0.1% trifluoroacetic acid. Final peptide concentrations were determined with Ellman's reagent, and cyclic peptide yields ranged 20-71%. See FIG. 7D.

Ribonucleotide Reductase Expression and Purification

Large subunit (mR1) gene cloned into pET28a was transformed into E. coli BL21(λDE3) Rosetta™ (Novagen) for overexpression, and small subunit (mR2) gene cloned into pET28a was transformed into E. coli BL21(λDE3) (Novagen) for overexpression (see Example 2). Briefly, 10 mL of overnight cultures were inoculated into a 2 L flasks containing 1 L LB supplemented with appropriate antibiotics. The cultures were grown with shaking at 250 rpm at 37° C. until OD₆₀₀=0.6, and then temperature was shifted to 18° C. Expression of both mR1 and mR2 were induced with 1 mM IPTG, and the cultures were incubated for another 24 hr at 18° C. Cells were pelleted in a Sorvall 5RCB+ centrifuge with a GS3 rotor at 6000 rpm for 10 min.

The pellets were resuspended in 40 mL of binding buffer (20 mM sodium phosphate, 500 mM NaCl, pH 7.8) with one tablet of Complete™ Protease Inhibitor Cocktail lacking EDTA (Roche). Lysozyme was added to 1 mg/mL and the suspensions were incubated on ice for 30 min. Triton X-100 (1%) and DNase (5 μg/mL) were added and the mixtures were incubated on a rocking platform for 10 min at 4° C. Insoluble debris was removed by centrifugation at 16,000 rpm in a Sorvall SS-34 rotor for 30 min at 4° C. The cleared lysates were applied TALON Metal Affinity Resin (BD Biosciences) and purifications were performed according to manufacturer's instructions. Protein fractions were pooled, concentrated using Amicon Ultra-15 centrifugal filter device (Millipore), and dialyzed into 50 mM Tris, 100 mM NaCl, 1 mM DTT, pH 8.0. Typical yield were 10-15 mg of both proteins per liter of culture.

Thermodynamic Dissociation Constants

The equilibrium dissociation constant, K_{D}, for His-tagged mR2·cRR130 complex was measured by the quenching of intrinsic protein fluorescence as a function of ligand concentration using a Flouromax-2 (SA Instruments) spectrofluorometer. His₆-mR2 was added to 1× phosphate buffered saline (PBS) buffer at pH 7.0, and enzyme concentrations were kept below the K_{D} being measured and were typically 1 μM. The small subunit mR2 contains six tryptophan residues whose combined fluorescence was monitored at 350 nm with excitation at 295 nm. Fluorescence data were collected as a function of added cRR130. The data was corrected for ligand background fluorescence and were fit to a hyperbolic equation to generate the K_{D} value.

ELISA Methods

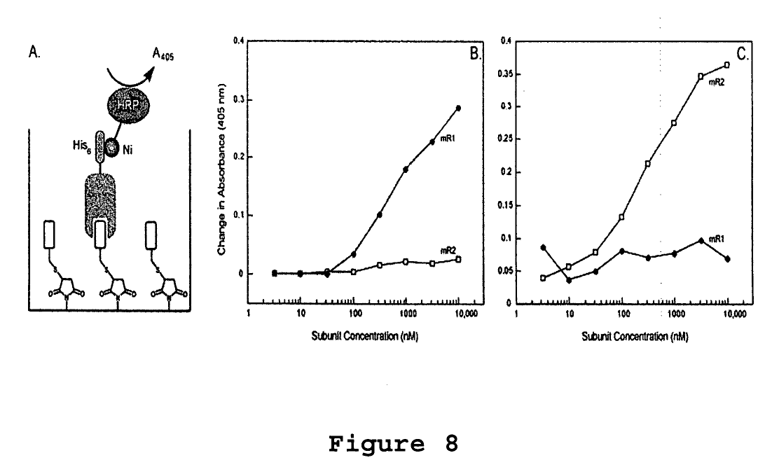

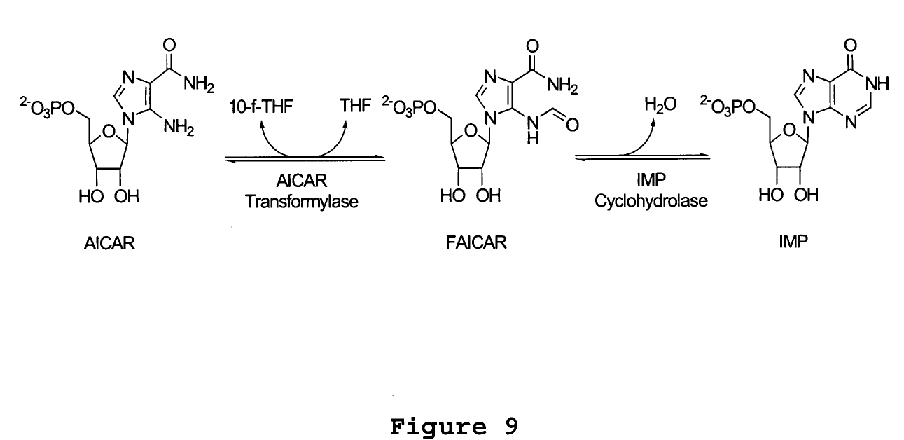

Two variations of solid phase binding assays were used for analyzing the binding of peptide inhibitors to ribonucleotide reductase subunits: i) protein competition ELISA where peptides were competing with mR1 for binding to immobilized mR2, ii) binding and competition ELISA with covalently immobilized ligands. In general, the solid phase assays were performed in microtiter plates (MaxiSorp, Nunc) or strip units (Reacti-Bind™ Maleimide Activated Clear Strip Plates, Pierce) involving continuous agitation in Junior Orbit Shaker (Lab-line Instruments) at medium speed during all of the incubation steps. Sample volumes were 100 μL, unless specified otherwise. Following coating, blocking step was conducted by incubating pre-loaded wells with 5% bovine serum albumin in PBS for 1 h at room temperature. Wash procedures between any two successive incubations involved three washes with 200 μL of 0.5% Tween-20 in PBS (PBST), with the second wash involving a 5 min incubation. Detection of His-tagged proteins was performed with Ni·NTA-HRP conjugate (Qiagen) according to the manufacturer instructions. See FIG. 8A.

Dissociative mR2·mR1 ELISA

Competition ELISA was performed with mR2 coated overnight (at 4° C.) onto MaxiSorp 96-well microtiter plates at a concentration of 50 μg/ml in 50 mM carbonate-bicarbonate buffer (pH 9.6). Following the blocking step, the wells were exposed to undersaturating amounts of His-tagged mR1 (typically 0.06 μM) with or without inhibitors. Retained mR1 was detected via Ni·NTA-HRP conjugate. See FIGS. 8B and 8C.

Peptide Binding/Competition ELISA

Peptides (200 nmol per well) in 10% DMF/50 mM Tris·HCl (pH 7.5) with 1 mM TCEP were reacted for 2 h at room temperature with maleimide-derivatized polystyrene wells. The unreacted sites were blocked by incubating wells with 5 μM cysteine in 50 mM Tris·HCl (pH 7.5) for 30 min. Following washing and blocking steps, the wells were incubated with His-tagged mR1 or mR2 in the presence or absence of inhibitors. The retained protein was detected via Ni·NTA-HRP conjugate.

### EXAMPLE 2

### DNAs, Bacterial Strains and Selections

Materials.

All reagents were purchased from VWR or Sigma Chemical. Restriction enzymes and polymerases were purchased from New England Biolabs. Oligonucleotides were synthesized on a 8909 Perceptive Biosystems Expedite DNA synthesizer. Linear peptides were synthesized at Hershey Macromolecular Core Facility of Pennsylvania State University. Plasmid, PCR purification, and gel extraction kits were purchased from Qiagen.

Recombinant DNA Techniques

E. coli cultures were maintained in Luria-Bertani (LB) broth. DNA manipulations were performed with E. coli DH5α-E (Invitrogen) or DH5αpir cells Platt, R., et al. (2000) Plasmid 43:12-23]. Plasmids were transformed into E. coli by heat-shock or electroporation [Inoue, H., et al. (1990) Gene 96:23-8]. All DNA sequencing was performed at the Nucleic Acids Facility of Pennsylvania State University.

Plasmid Constructions:

A. Triple Reporter Cassette

The HIS3 gene was PCR amplified from Saccharomyces cerevisiae genomic DNA and ligated into the BamHI and SacI sites of pSU19 [Bartolome, B., et al. (1991) Gene 102:75-8]. The kanamycin resistance gene was PCR amplified and ligated into SacI and EcoRI pBAD18 [Guzman et al. (1995) J. Bacteriol. 177:4121-3410]. The GFP reporter gene was cloned flanking to the kanamycin resistance (Kan^{R}) gene in pBAD18 using AatII and SacII sites, which were incorporated into the Kan^{R} gene 3′ primer. To generate the HIS3-Kan^{R}-GFP triple reporter, the Kan^{R}-GFP cassette was cloned downstream of the HIS3 gene in pSU19 using SacI and EcoRI sites. Wild-type phage 434 and 434-P22 chimeric promoter regions were generated with overlapping oligonucleotides and cloned into the PstI and XbaI sites of the HIS3-Kan^{R}-GFP triple reporter plasmid. Each step of the construction process was verified by sequencing, and the entire triple reporter cassette was removed with SphI and HpaI and cloned into SphI and AflII (blunted) sites of pCD13PKS [Platt et al. (2000) Plasmid 43:12-23]. Upon integration, the GFP expression level was not adequate for quantitative analysis, prompting replacement of the GFP marker with the lacZ gene from plasmid pAH125 [Haldimann et al. (2001) J. Bacteriol. 183:6384-6393].

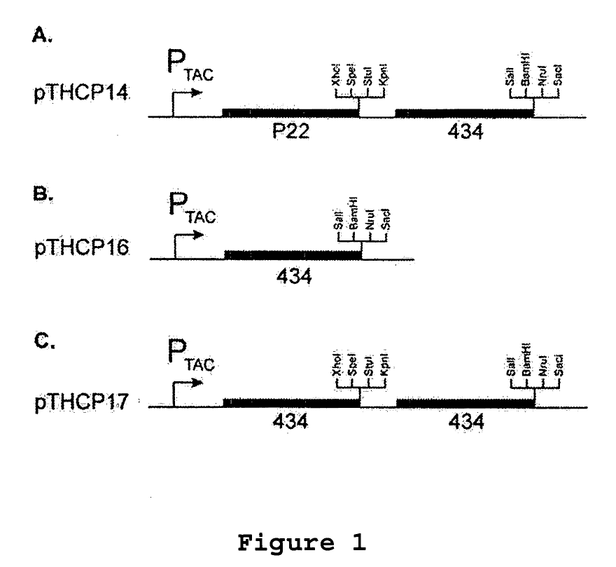

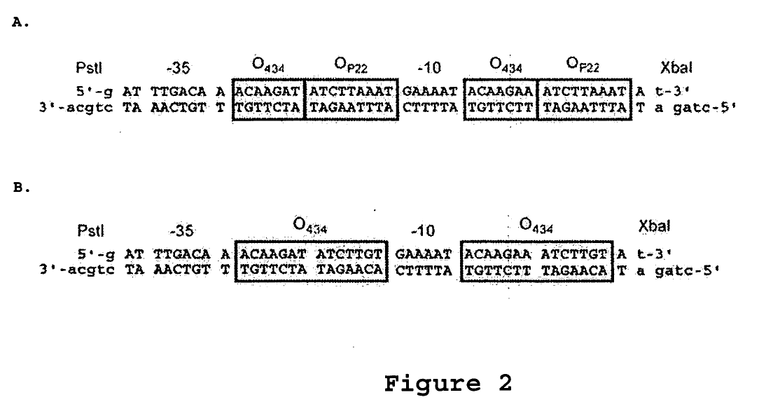

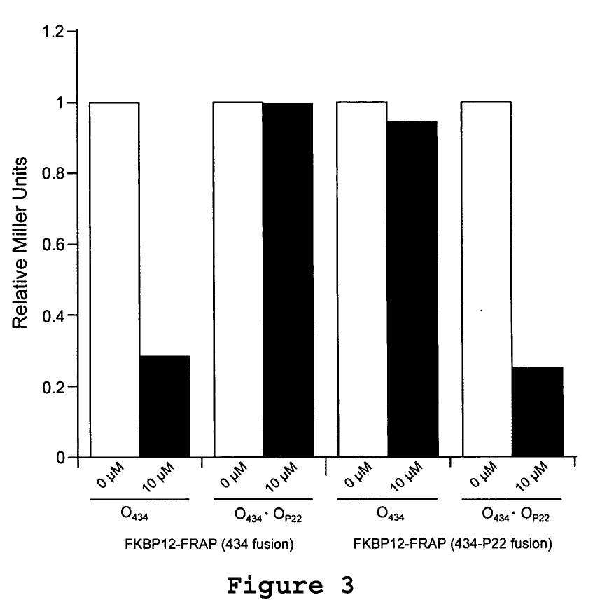

Fusion Cloning Constructs (See FIGS. 1, 2, 6):

pTHCP14. (FIG. 1A) An inducible plasmid containing the DNA-binding domains of both wild-type 434 repressor and a mutant 434 repressor with P22 specificity (hereafter referred to as P22 repressor) was constructed in a similar fashion as previously described [Di Lallo et al. (2001) Microbiology 147:1651-1656]. The resulting plasmid contains an IPTG-inducible P_{TAC} promoter and vector backbone from pMAL-c2x (New England Biolabs) and different restriction sites for creating C-terminal fusions.

pTHCP16. (FIG. 1B) A second plasmid containing only 434 repressor was constructed.

pTHCP17. (FIG. 1C) A third plasmid containing tandem copies of 434 repressor with orthogonal cloning sites was constructed.

Repressor Control Construction:

pTHCP12. Wild-type 434 repressor cloned into pMAL-c2x.

pTHCP15. Wild-type 434 and P22 repressors cloned in tandem into pMAL-c2x.

pTHCP20. S. cerevisiae GCN4 transcription factor was PCR amplified from plasmid pJH370 [Hu et al. (1990) Science 250:1400-1403] and cloned into SalI and BamHI sites of pTHCP16.

Fusion Constructs: FRAP & FKBP12 (Rapamycin-Binding)

pTHCP25. Human FRAP residues 2018-2112 (rapamycin binding domain) were PCR amplified from placenta cDNA library (Clontech) and cloned into SalI and SacI sites on pTHCP14. Human FKBP12 was PCR amplified from the same cDNA library and cloned into the pTHCP14-FRAP plasmid at the XhoI and KpnI sites.

pTHCP26. FRAP and FKBP12 were cloned into pTHCP17 in same manner as described for pTHCP25.

Fusion Constructs: Ribonucleotide Reductase

pTHCP30. Murein ribonucleotide reductase subunit R1 was PCR amplified from a Bacuolovirus expression plasmid [Caras et al. (1985) J. Biol. Chem. 260:7015-7022] and cloned into SalI and SacI sites on pTHCP14, and subunit R2 was PCR amplified from pET3a-R2 and cloned onto pTHCP14-R1 plasmid at XhoI and KpnI sites [Mann et al. (1991) Biochemistry 30:1939-1947].

pTHCP32. Ribonucleotide reductase was removed from pTHCP30 using BsaBI and SacI and cloned into pAH68 [Haldimann et al. (2001) J. Bacteriol. 183:6384-6393] digested with HincII and SacI.

Fusion Constructs: HIV Protease

pHIV5. HIV-1 protease was PCR amplified from pET-HIV-1 [Ido et al. (1991) J. Biol. Chem. 266:24359-24366] and cloned into SalI and BamHI sites of pTHCP16. The catalytic aspartate (D25) was mutated to asparagine using 3-primer PCR [Michael (1994) Biotechniques 16:410-412], and a S(G)₄S linker was added at the SalI site.

Inhibitor Constructs: Controls

pTHCP35:

Overlapping oligonucleotides encoding MSFTLDADF (methionine plus eight R2 subunit C-terminal residues) (SEQ ID:15) were cloned into NcoI and XbaI sites on arabinose expression plasmid pAR [Perez-Perez et al. (1995) Gene 158:141-142].

pTHCP37

Overlapping oligonucleotides encoding MDTAFSFLD (scrambled peptide control) (SEQ ID:16) were cloned into NcoI and XbaI sites on pAR.

pHIV16

Overlapping oligonucleotides encoding MTVSYEL (methionine plus hexapaptide control inhibitor) (SEQ ID:17) [Schramm et al. (1996) Antiviral Res. 30:155-170] were cloned into EcoRI and SphI sites on arabinose expression plasmid pBAD18.

pHIV17

Overlapping oligonucleotides encoding MDSATYV (methionine plus control peptide) (SEQ ID:18) were cloned into EcoRI and SphI sites on pBAD18.

Strain Constructions

E. coli strain BW27786 was used for all genetic selections [Khlebnikov et al. (2001) Microbiology 147:3241-3247]. Residues 1-164 of HisB corresponding to the imidazole glycerol phosphate dehydratase activity were deleted on the chromosome of strain BW27786 using the phage λ Red system [Datsenko et al. (2000) Pro. Nat. Acad. Sci. 97:6640-6645]. Integration of the triple reporter and repressor fusions was performed as previously described [Platt et al. (2000) Plasmid 43:12-23; Haldimann (2001) J. Bacteriol. 183:6384-6393]. Strain BW27786 ΔhisB with homodimeric (FIG. 1C) reporter (HIS3-Kan^{R}-lacZ operon) was designated SNS118 and the heterodimeric (FIG. 1A) reporter was designated SNS126.

Library Constructions

SICLOPPS libraries were constructed on pAR-CBD vector as previously described [Abel-Santos et al. (2003) Methods Mol. Biol. 205:281-294]. C+5 libraries were constructed by altering previously utilized peptide scaffolds [Scott et al. (2001) Chem. Biol. 8:801-815].

Mock Selection

Ribonucleotide reductase repressor fusions were moved to pAH68 and integrated into SNS126 as described [Haldimann et al. (2001) J. Bacteriol. 183:6384-6393]. Plasmids pTHCP35 and pTHCP37 were mixed at 1:100 ratio, and this mixture was transformed into the ribonucleotide reductase repressor strain. The transformants were plated at a density of 10⁴ CFU/plate on minimal media supplemented with 2.5 mM 3-AT, 50 μg/ml kanamycin, 200 μM IPTG, and 2×10⁻⁴% arabinose and incubated at 37° C. Colony PCR was performed on surviving colonies to ascertain the identity of the peptide sequence.

Ribonucleotide Reductase over Expression Constructs.

pET28a-MR1:

Ribonucleotide reductase subunit R1 was moved to pET28a (Novagen) from pTHCP30 using NheI and SacI sites.

pET28a-MR2:

Ribonucleotide reductase subunit R2 was moved to pET28a from pTHCP30 using BamHI and SacI sites.

pET28a-FKBP12:

FKBP12 was PCR amplified and cloned into NdeI and SacI sites on pET28a.

Culture Media and Growth Conditions

Antibiotic concentrations were provided at the following concentrations: ampicillin, 100 μg/ml; chloramphenicol, 50 μg/ml; kanamycin, 50 μg/ml; spectinomycin, 50 μg/ml; tetracycline, 20 μg/ml. For chromosomal markers, concentrations of antibiotics were reduced two-fold. Minimal media A (MMA) supplemented with 0.5% glycerol and 1 mM MgSO4 was used for genetic selections.

Genetic Selections

SICLOPPS libraries were transformed into E. coli strains containing integrated reporter and repressor constructs. Transformants were washed with minimal media A and plated on minimal media supplemented with 2×10⁻⁴% L-(+)-arabinose and 3-AT, kanamycin, and IPTG concentrations determined for optimal stringency. Following incubation at 37° C. for 3-4 days, surviving colonies were restreaked onto the same media with and without arabinose. Plasmids from selected strains, whose growth was dependent on the presence of arabinose, were retransformed into the original selection strain and checked for phenotype retention. The variable insert regions on SICLOPPS plasmids were PCR amplified and their DNA sequence was determined.

### EXAMPLE 3

### Bacterial RTHS

Overall Design Strategy

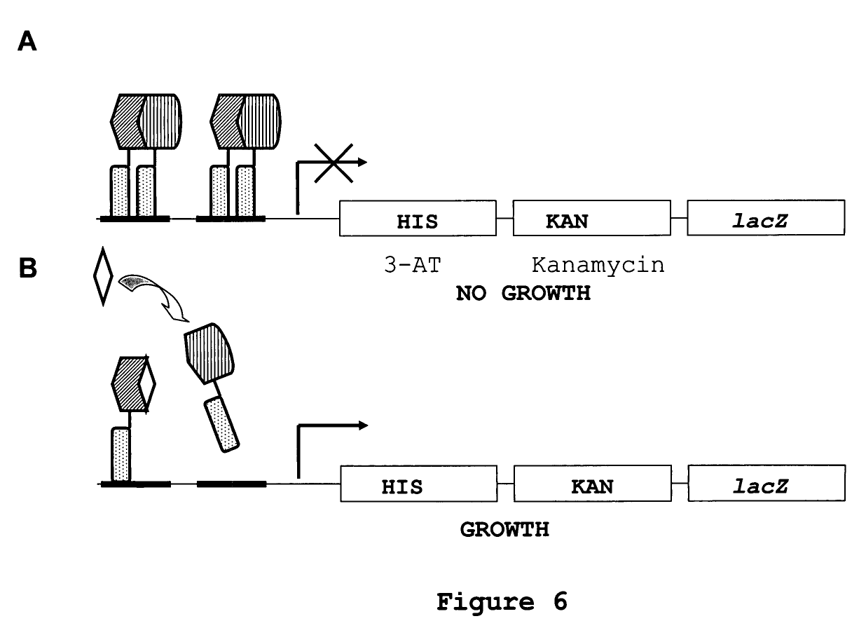

A bacterial version of the RTHS that functions in parallel with SICLOPPS was designed. This approach greatly enhanced the throughput capacity and drew on the successful implementation of SICLOPPS in Escherichia coli [Scott et al. (2001) Chem. Biol. 8:801-815; Scott et al. (1999) Pro. Nat. Acad. Sci. 96:13638-13643]. As depicted in FIG. 6, the design was based on the bacteriophage repressor and features a positive genetic selection, which is less likely to yield false positives resulting from RTHS-independent effects on growth rates.

The RTHS design adapted elements from several bacterial systems to create a robust, flexible, and tunable genetic selection for molecules that modulate protein-protein interactions. The key features of this system are as follows: i) chimeric repressors to monitor true heterodimeric interactions [Di Lallo et al. (2001) Microbiology 147:1651-1656]; ii) two conditionally selective reporters, HIS3 [Joung et al. (2000) Pro. Nat. Acad. Sci. 97:7382-7387; Brennan et al. (1980) J. Mol. Biol. 136:333-338] (imidazole glycerol phosphate dehydratase) and Kan^{R} (aminoglycoside 3′-phosphotransferase for kanamycin resistance), to allow synergistic selections [Stavropoulos et al. (2001) Genomics 72:99-104] and chemical tunability; and iii) LacZ (β-galactosidase) for quantitative measurements of protein-protein interactions. Further details on constructions, reporters, and strains are provided in the previous Examples.

Validation of Reporter and Repressor Design

The ability of the RTHS to report on protein complex formation was investigated with a number of model systems. The wild-type 434 repressor protein was used, as well as DNA-binding domain fusions with S. cerevisiae GCN4 leucine zipper, and HIV-1 protease to monitor homodimeric interactions, and fusions with murine ribonucleotide reductase subunits as an example of a heterodimeric complex. β-galactosidase activity assays documented levels of protein-protein interactions in reporter strain SNS118 expressing DNA-binding domain (negative control), 434 repressor (positive control), GCN4 transcription factor, and HIV-1 protease, respectively at IPTG concentrations of zero, 10 μM, and 50 μM, as listed in Table 2.

TABLE 2
β-glycosidase activity in Relative Miller Units
(RMU) as a function of IPTG concentration

| | | | GCN4 | |
|---|---|---|---|---|
| IPTG | negative | positive | transcription | HIV-1 |
| conc'n | control | control | factor | protease |
| none | 1.00 | 0.10 | 0.20 | 0.92 |
| 10 μM | 0.95 | 0.12 | 0.16 | 0.94 |
| 50 μM | 1.02 | 0.12 | 0.15 | 0.23 |

β-galactosidase activity assays documented levels of protein-protein interactions in reporter strain SNS126 with integrated null (negative control), ribonucleotide reductase, and FKBP12-FRAP fusions (with and without rapamycin 1 μM) as a function of IPTG concentrations: zero, 20 μM, 650 μM. See Table 3.

TABLE 3
β-Galactosidase Activity in
Relative Miller Units (RMU)

| | | | FKBP12- | |
|---|---|---|---|---|
| IPTG | | | FRAP | FKBP12-FRAP |
| conc'n | negative | Ribonucl. | fusion + | fusion − |
| (μM) | control | reductase | rapamycin | rapamycin |
| none | 1.00 | 0.72 | 0.70 | 1.00 |
| 20 | 0.98 | 0.45 | 0.34 | 1.40 |
| 650 | 0.90 | 0.20 | 0.30 | 0.92 |

The fusion constructs, therefore, repressed the lacZ reporter approximately 4-9 fold, a dynamic range typical of other repressor-based systems [Di Lallo et al. (2001) Microbiology 147:1651-1656].

To visualize the effect on cell growth on media containing kanamycin, and to visualize the β-galactosidase activity using X-GAL as chromogenic indicator, drops of rapamycin solution were applied at 1, 3, 10, 30, 100, 300 μM concentrations on cell lawns containing integrated FKBP12-FRAP fusions. Growth and β-galactosidase activity were slightly inhibited by 3 μM and 10 μM, and very inhibited by 100 μM and 300 μM rapamycin.

By examining β-galactosidase activity as a function of rapamycin concentration, it was found that IC₅₀=209 nM (±31), that is, 209 nM rapamycin inhibited β-galactosidase activity by 50%.

Together these results demonstrate that the fusion proteins and their interactions did control growth and β-galactosidase activity in a manner that was readily observed both quantitatively and qualitatively (by eye).

Control Peptide Inhibitors

The initial RTHS efforts were focused on two well characterized model systems, homodimeric HIV-1 protease and heterodimeric ribonucleotide reductase, whose enzymatic activities are dependent on subunit association. These enzymes are attractive targets due to their importance for HIV-infection and cancer proliferation, respectively, and their recent status as representatives of multi-disciplinary efforts to disrupt both homo- and heterodimeric protein-protein interfaces [Cochran (2000) Chem. Biol. 7:R85-94; Berg (2003). Angew. Chem. Int. Ed. 42:2462-2481]. Interference with such complexes has been proposed as a superior alternative to chemotherapies targeting enzymes' active sites, due to the intrinsically higher specificities and lower resistance frequencies associated with this approach [Zutshi et al. (1998) Curr. Opin. Chem. Biol. 2:62-66].

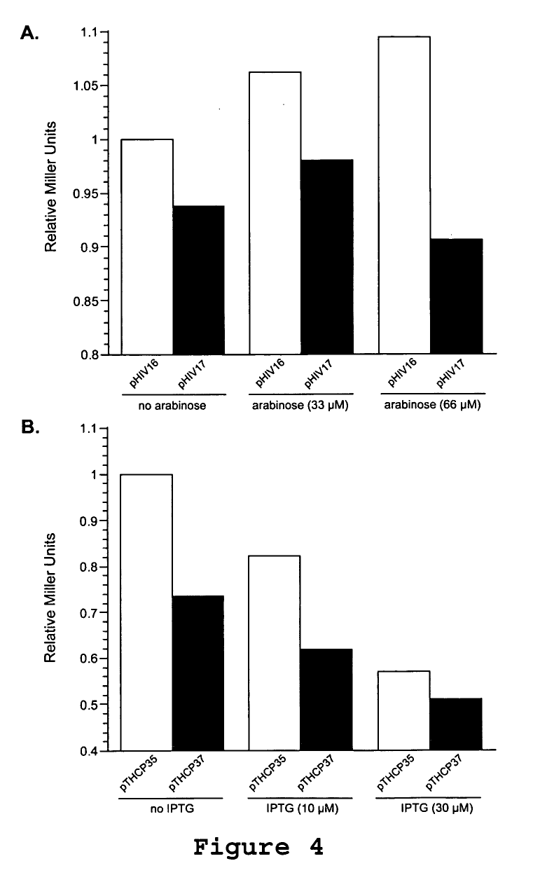

Towards this goal, both enzyme complexes were probed with known linear peptidic inhibitors. These peptides are a C-terminal hexapeptide (FIG. 4A, pHIV16) for HIV protease that inhibits the essential β-sheet interactions [Schramm al. (1996) Antiviral Res. 30:155-170], and a heptapeptide for ribonucleotide reductase (pTHCP35, FIG. 4B) that competes with binding between subunits mR1 and mR2 [Yang et al. (1990) FEBS Lett. 272:61-64]. When co-expressed with target fusions, the inhibitor peptides, and not scrambled controls, relieved repression of the lacZ reporter (FIG. 4), validating the system design as well as demonstrating the potential for selections based on effectors that cause or stabilize intra-macromolecule interactions.

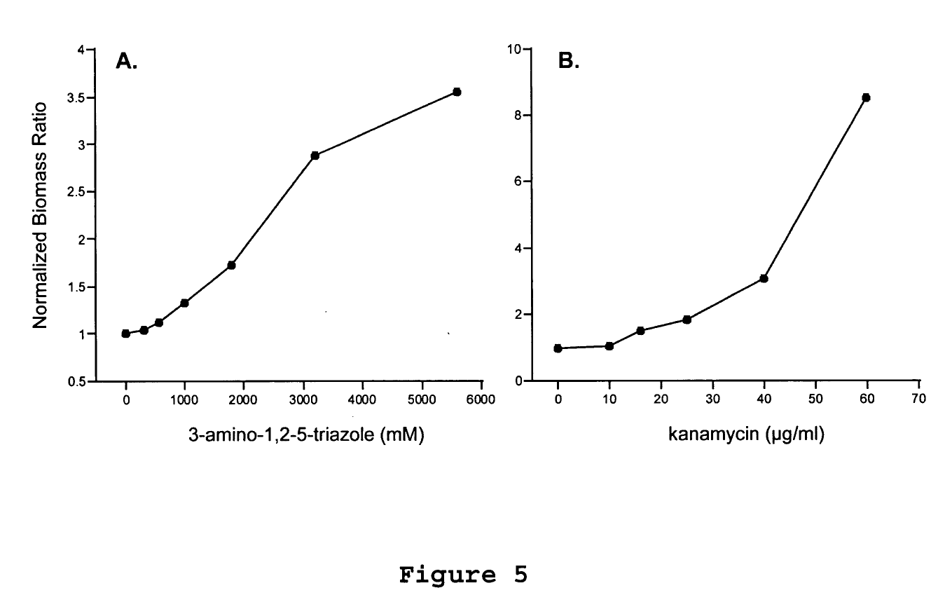

For genetic selection to yield molecular candidates within large populations, the growth advantage of the selectants should be maximized. In the course of these studies, the advantage of utilizing tunable reporters became apparent when inadvertent background expression was overcome by titrating reporter activities with the chemical agents 3-amino-1,2,5-triazole (3-AT) and kanamycin (FIG. 5).

Once the selection parameters had been fully explored, the ability of the RTHS to discriminate between candidates with a range of dissociative activities was investigated in a mock selection format. For this purpose, populations expressing scrambled control peptides were spiked with plasmids encoding a known ribonucleotide reductase inhibitor. By exclusively retrieving the inhibitor expressing strains, the advantages provided by our RTHS design, such as i) positive selection format; ii) synergistic reporter effects; iii) chemical tunability, lay the groundwork for the identification of novel inhibitors from libraries.

### EXAMPLE 4

### Peptidyl Modulators of Ribonucleotide Reductase

As a case study, ribonucleotide reductase fusions were tested against SICLOPPS libraries with an intent to discover cyclic peptides acting as dissociative inhibitors. Predictions from modeling studies [Gao et al. (2002) Bioorg. Med. Chem. Lett. 12:513-515] suggested that the reverse turn conformations of known complex disruptors should be well represented within SICLOPPS libraries.

A library, encoding hexapeptides with five random residues and an invariable cysteine as a cyclization nucleophile, was transformed into the RTHS E. coli strain expressing ribonucleotide reductase fusions. The transformants were plated on selective media (histidine-free minimal media supplemented with 3-AT and kanamycin) at a density of 10⁶-10⁷, from libraries containing up to 10⁸ individual plasmids. The plates were incubated until readily identifiable colonies (about one in 10⁵ for ribonucleotide reductase) could be collected and processed further to confirm a relationship between growth advantage and SICLOPPS plasmid expression, thus eliminating false positives.

This relationship was observed by comparing the growth rates on selective media with and without arabinose induction of SICLOPPS expression, which resulted in approximately 90% of isolates being false positives (data not shown). The individuals with enhanced survival rates that no longer correlated with induction were eliminated from further consideration.

To identify superior candidates, serial dilutions of cells expressing the peptides were spotted on selective plates, which permitted growth trends to be compared at each dilution level. The candidates showing dependence on arabinose induction and superior growth enhancement were advanced to further characterization to confirm their mode of action. The one in a million success rate underscores the challenges in the discovery of modulators of protein-protein interactions, an undertaking that has been described as “genuinely difficult” [Cochran (2000) Chem. Biol. 7:R85-94], and further outlines the importance of having a methodology capable of high-throughput.

One of the principle advantages of genetically encoded combinatorial libraries is the ease of deciphering their chemical composition, in contrast to synthetically derived libraries. Thus, the amino acid sequence was readily determined for each candidate by DNA sequencing of the variable inserts present on the selected SICLOPPS plasmids. The sequences of the most potent in vivo selectants (FIG. 7A) can be tentatively grouped into neutral and charged consensus classes. Remarkably, neutral class motifs resemble the Ar-X-F sequence (where Ar is an aromatic amino acid; X is any amino acid) identified previously for linear dissociative inhibitors of ribonucleotide reductase [Gao et al. (2002) Bioorg. Med. Chem. Lett. 12:513-515]. Surprisingly, the C-terminal negative charge documented to be critical for recognition of large enzyme subunit was absent in all identified sequences.

As a secondary test to assess selectivity, the peptides were challenged with a control target fusion. For the 8 candidates presented in FIG. 7A, five linear peptides (l-RR84, l-RR93, l-RR112, l-RR127, and l-RR130) showed more than 100-fold growth enhancement for ribonucleotide reductase over the control RTHS (data not shown), presumably by blocking the association of the reductase subunits.

To address non-specific effects of selectants on host growth rates, three of the selective peptides (RR93, RR127, RR130) and the less discriminating RR133 were subjected to quantitative expression studies using the lacZ reporter of the RTHS. All four peptides showed observable repression relief with background level of expression, and for three of the peptides (RR93, RR127, RR133) the effect was further enhanced upon arabinose induction (FIG. 7B). Surprisingly the fourth selectant, RR130, triggered arabinose-dependent repression of the lacZ reporter, suggesting a complex mode of action. These studies necessitated in vitro analysis to decipher the inhibition mechanism of these four selectants.

The absence of a dissociative assay for ribonucleotide reductase prompted the development of a screen based on the competition enzyme-linked immunosorbent assay (ELISA). This procedure involves the immobilization of the small subunit (mR2) on a polystyrene surface, followed by its specific recognition with the His-tagged large subunit (mR1), whose presence is detected by a nickel-nitrilotriacetic acid-horse radish peroxidase (Ni-NTA-HRP) conjugate reacting with the 2,2′-azinobis[3-ethylbenzothiazoline-6-sulfonic acid] (ABTS) chromogenic substrate (detected as absorbance change at 405 nm). The activity of inhibitors can be monitored by their concentration-dependent reduction in a HRP-dependent signal due to the disruption of the complex.

The synthetic linear peptides corresponding to the four genetically selected sequences promoted dissociation of the immobilized complex as shown in FIG. 6C. This finding generally matched the in vivo observed trends, and peptide l-RR93 showed the most activity. Although none of these peptides surpassed the potency of the C-terminal octapeptide control (P8), all functioned as dissociative inhibitors in the in vitro assay. This demonstrates the power of genetic selection to identify rare solutions to the problem of inhibiting protein complexation. Moreover, the cyclization of these peptides conformationally restricts presentation of the active epitope, and thus improves their potency.

Due to the challenges inherent to peptide head-to-tail backbone cyclization, a novel solid phase strategy was devised exploiting immobilization of linear sequences through a cysteine side chain as a mixed disulfide (FIG. 7D). This approach was expected to favor monomolecular cyclization over bimolecular side reactions, due to a solid phase dilution effect. In addition to other advantages of solid phase synthesis, such as improved yield and ease of purification, the disulfide immobilization strategy permits convenient isolation of the product via mild reductive cleavage with suitable thiol or phosphine reagents. Using this approach, all four linear peptides (l-) under investigation (l-RR93, l-RR127, l-RR130, l-RR133) were cyclized and their chemical nature was confirmed by a combination of Kaizer assay [Kaiser et al. (1970) Anal. Biochem. 34:595-598], reverse-phase HPLC, and electrospray ionization (ESI) mass spectrometry (Table 4).

TABLE 4
Mass spectrometry results and synthesis yields
of cyclic peptides

| Peptide | Mass (calc) | m/z (obs) | % Yield |
|---|---|---|---|
| c-RR93 | 804.3 | 804.1 | 20 |
| c-RR127 | 710.3 | 710.2 | 61 |
| c-RR130 | 799.4 | 799.3 | 57 |
| c-RR133 | 806.4 | 806.4 | 71 |

The cyclized peptides were assayed against immobilized ribonucleotide reductase complex in the dissociative ELISA assay (data not shown). Compared to their linear forms, both cyclic RR93 and RR127 (c-RR93 & c-RR127) exhibited an approximately 2-fold enhanced activity over the corresponding linear forms, confirming the entropic benefits of a constrained scaffold. The dissociative ELISA could not confirm the properties of less specific c-RR130 and c-RR133, yielding a response pattern consistent with nonspecific adherence to plastic surface.

Despite the precedent for inhibitors based on the C-terminus of mR2 [Yang et al. (1990) FEBS Lett. 272:61-64], the functional nature of the genetic assay implies that peptides targeting either surface (mR1 or mR2) are capable of perturbing the repressor complex. Although confirming the dissociative properties of the four selected sequences, the functional format of the protein ELISA, relying merely on the complex disruption for read-out, is incapable of unambiguously identifying the receptor for the peptide ligands.

To determine the mechanism of action of the four identified sequences an alternative assay format was devised (FIG. 8A), where a peptide with a residual activity in its immobilized form can serve as a specific ligand for receptor capture in both binding and competition ELISA. The success of such an assay relies on both efficient peptide immobilization strategy and sufficient level of affinity, uncompromised by this display strategy. The peptide immobilization becomes feasible through implementation of a cysteine, a nucleophile used in splicing, as a universal chemoselective handle allowing covalent attachment strategy through a suitable electrophile. Chemoselective attachment of such peptides on appropriately derivatized surfaces should both display the small molecules for detection with a suitable protein receptor and allow binding site competition analysis, not unlike in a traditional immunosorbent format.

When immobilized on maleimide plates via its N-terminal cysteine, the P8 peptide maintained its residual specific affinity toward mR1 (FIG. 8B). Moreover, the resulting P8·mR1 immobilized complex can be disrupted by both l-RR93 and c-RR93 (linear and cyclic peptide RR93, respectively) in a concentration dependent manner, with activity profiles comparable to those from the protein ELISA (not shown). These results point to direct competition for the common binding site on mR1 by both the C-terminus of mR2 (P8) and the selected RR93 sequence. The presence of the Ar-X-Ar motif in RR93 and other selectant sequences (e.g., RR84 and RR120) is consistent with the previously documented importance of this motif in targeting the mR2 subunit [Gao et al. (2002) Bioorg. Med. Chem. Lett. 12:513-515].

The fact that none of the positively charged sequences (RR127, RR130, and RR133) retained mR1, when immobilized or competed with P8·mR1 complex (data not shown), suggested an alternative, perhaps, common mode of ribonucleotide reductase complex disruption. A systematic analysis of immobilized linear and cyclic forms yielded an unexpected observation that, unlike P8, c-RR130-derivatised surface selectively captured His₆-tagged mR2 subunit, while being immune to His₆-tagged mR1, when exposed to the increasing protein concentrations (FIG. 8C). Thus, binding partners for both small and large ribonucleotide reductase subunits were identified, demonstrating the capacity of genetic selection to discover not only novel inhibitors but, importantly, new targets.

Furthermore, confirming the original putative division of the selectants into charged and neutral categories, all three positively charged peptides (i.e., RR127, RR130, and RR133) competed with the c-RR130·mR2 complex (data not shown), with activities generally consistent with the protein ELISA observations. Thus, l-RR133 showed the highest capacity in dislodging mR2 from the cyclic peptide anchor, followed by l-RR130, and both forms of RR127. Although both c-RR130 and c-RR133 proved again to be incompatible with the ELISA due to, presumably, nonspecific adsorption, their K_{D} values were determined by quenching of intrinsic mR2 tryptophan fluorescence to be 53 μM (±5) and 133 μM (±42), respectively (data not shown). The activities of the corresponding linear counterparts were significantly lower in the fluorescence-quenching assay, precluding their thermodynamic characterization, due to the peptide fluorescence interference and solubility limits. These observations point again to the reduction of a conformational population by constraining flexible molecules as means of improving activity of protein modulators.

### EXAMPLE 5

### Rapamycin-Dependant Modulation

The chemical modulation of a protein-protein interaction in an exemplary RTHS was also demonstrated with the FKBP12 (FK506 binding protein) and FRAP (FKBP12-rapamycin associated protein) pairing, whose dimerization is dependent on the presence of rapamycin [Brown et al. (1994) Nature 369:756-758], a naturally occurring chemical dimerizer. As discussed above, cell growth and β-galactosidase assays demonstrated that rapamycin was taken up by E. coli triggering the assembly of a functional repressor composed of heterologously expressed FKBP12 and FRAP fusions.

Rapamycin functioned in a concentration dependent manner with an IC₅₀ of 209 nM (±31). Similarly, varying the levels of FKBP12 and FRAP at fixed rapamycin concentrations correlated with the levels of β-galactosidase activity (data not shown).

Small molecule-dependent modulation as well as a satisfactory dynamic range shows that method of the invention permits discovery of molecules that promote as well as molecules that interfere with protein-protein contacts when genes directing their synthesis are present in cells containing such reporter systems.

### EXAMPLE 6

### In Vivo Screening of Rapamycin Analogues

Rapamycin analogues can be prepared in vivo in two ways: biosynthetic genes can be mutated [Khaw et al., (1998) J. Bact. 180:809-814; Del Vecchio et al., (2003) J. Ind. Microbiol. and Biotechnol. 30:489-494] or the bacteria can be fed or caused to synthesize particular precursors [Graziani et al., (2003) Org. Lett. 5:2385-2388; Lowden et al., (2004) Chembiochem. 5:535-538]. A bacterial system for screening in vivo synthesized rapamycin analogues is made by insertion of the rapamycin polyketide gene cluster from Streptomyces hygroscopicus into the strain of E coli including the reporter gene system described above. Alternatively, the reporter gene system described above is inserted into the genome of an appropriate S. hygroscopicus strain.

In the case of E. coli, the rapamycin gene cluster can be subjected to in vitro mutagenesis by many well known techniques, including PCR mutagenesis, gene shuffling techniques, chemical or radiation treatment, etc., to prepare a library of mutant rapamycin gene clusters. This library is transformed into the reporter E. coli strain, which is then screened for increased rapamycin analogue-dependant gene expression.

In the case of S. hygroscopicus, the bacteria are subject to mutagenesis prior to introduction of the reporter gene cluster.

### EXAMPLE 7

### In Vivo Selection and Characterization of AICAR Tfase Inhibitors That Prevent AICAR Tfase Homodimerization

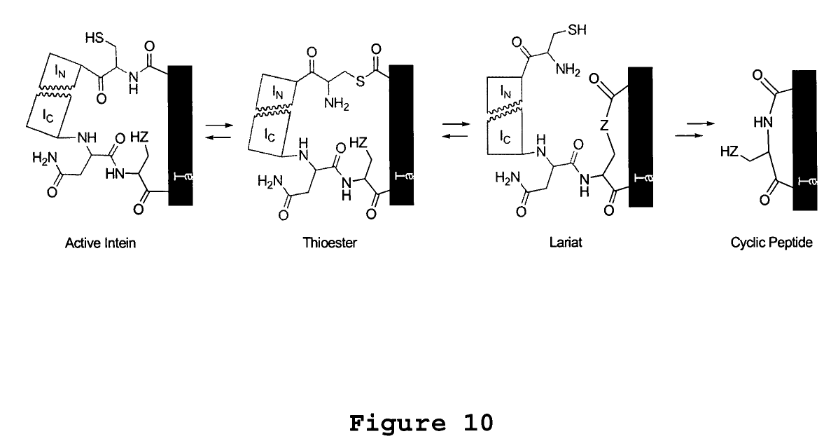

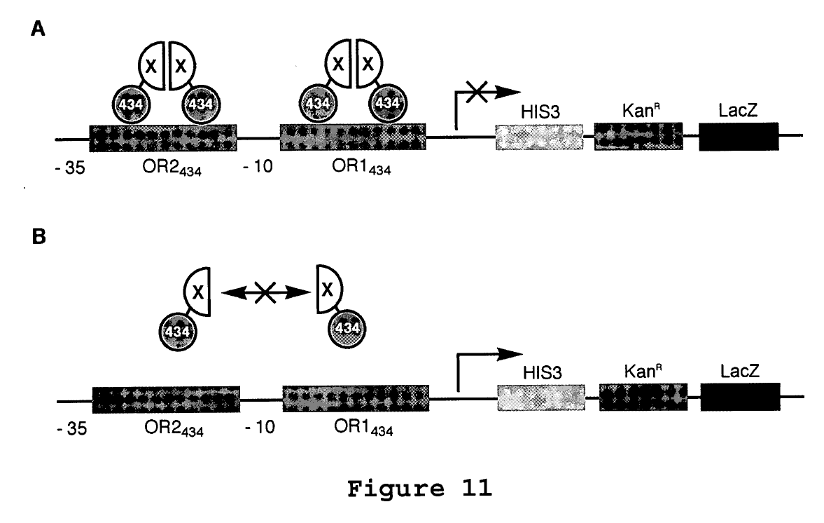

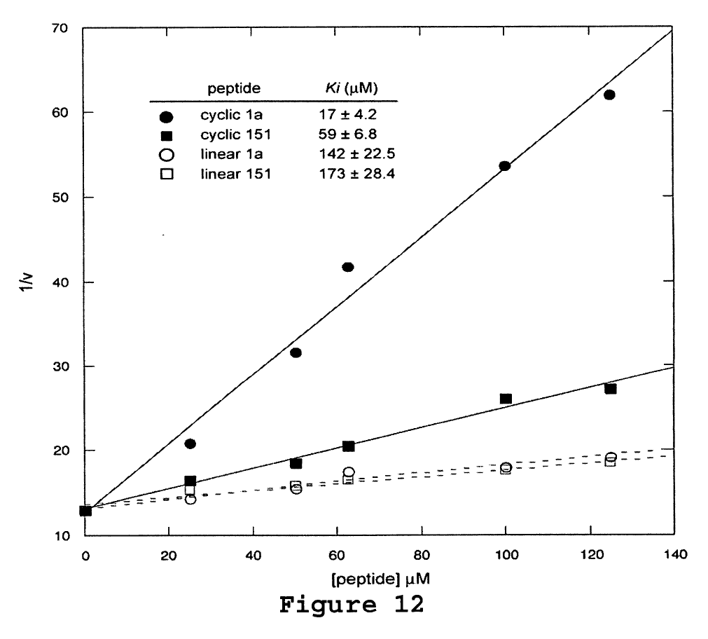

The de novo purine biosynthetic pathway is used by virtually all organisms for the production of purine nucleotides. The final two steps of this pathway (FIG. 10) are catalyzed by aminoimidazole-4-carboxamide ribonucleotide transformylase/inosine monophosphate cyclohydrolase (AICAR Tfase/IMPCH), the two activities of a highly conserved 64 kDa bifunctional protein (ATIC) possessing two distinct domains [Ni et al (1991) Gene 106:197]. The C-terminal AICAR Tfase domain (residues 200-593) catalyzes the transfer of a formyl group from N₁₀-formyl-tetrahydrofolate (10-f-THF) to AICAR. The N-terminal IMPCH domain (residues 1-199) catalyzes the final step of the pathway [Greasley et al. (2001) Nat. Struct. Biol. 8:402].

Cancer cells rely heavily on the de novo pathway for purine biosynthesis. Here, in vivo produced cyclic peptides are screened for their ability to specifically inhibit ATIC homodimerization and thereby inhibit AICAR Tfase activity [Jackson et al. (1981) Nucleotides and cancer treatment 18], thus inhibiting enzymes in this pathway is an attractive approach for development of anticancer agents. As well as their potential uses in the treatment of malignant diseases, ATIC inhibitors have uses in the treatment of inflammatory diseases such as rheumethoid arthritis [Gagdangi et al. (1996) J Immunol. 156:1937].

The AICAR Tfase activity of ATIC is dependent on its homodimerization, whereas the IMPCH activity is not. The recently reported crystal structure shows ATIC as a dimer with an interface of ˜5000 Å² [Greasley et al. (2001) Nat. Struct. Biol. 8:402]. There is much potential for the development of a new generation of therapeutic agents that act by inhibiting protein-protein interactions [Zutshi et al. (1998) Curr. Opin. Chem. Biol. 8:801]. We chose genetic selection as the means to identify small molecules that specifically inhibit ATIC homodimerization and thereby inhibit AICAR Tfase activity.

This example of a method of the invention utilizes whole cells as reporters of a designated intracellular event (interruption of a protein-protein interaction) by correlating host growth to the desired functional property of a small molecule. An advantage of this method is the selection of library members in vivo, allowing both affinity and selectivity to be assayed simultaneously.

Specifically the combination of our split intein-mediated circular ligation of peptides and proteins (SICLOPPS) technology (FIG. 10) [Scott et al. (2001) Chem. Biol. 8:801] with a bacterial reverse two hybrid system (RTHS) provides a method with the above characteristics for the systematic identification of small molecule inhibitors of protein-protein interactions [Horswill et al. (2004) Proc. Natl. Acad. Sci. USA 101:15591]. SICLOPPS allows the intracellular synthesis of libraries containing up to 10⁸ cyclic peptides, [Scott et al. (2001) Chem. Biol. 8:801] several orders of magnitude larger than that possible by conventional synthetic methods. Cyclization of peptides confers in vivo stability through their resistance to degradation by proteases [Tang et al. (1999) Science 286:498].

Our bacterial RTHS [Horswill et al. (2004) Proc. Natl. Acad. Sci. USA 101:15591] is based on the bacteriophage regulatory system [Hu et al. (1990) Science 250:1400] linking the disruption of the fusion protein homodimer to the expression of three reporter genes (FIG. 6). HIS3 [Joung et al. (2000) Proc. Natl. Acad. Sci. USA 97:7382] (imidazole glycerol phosphate dehydratase) and Kan^{R} (aminoglycoside 3′-phosphotransferase for kanamycin resistance) are two chemically tunable, conditionally selective reporter genes. The third reporter gene, LacZ (β-galactosidase) is used to quantify the protein-protein interaction through β-galactosidase assays.

ATIC was cloned as a fusion with the bacteriophage 434 repressor DNA binding domain (into pTHCP16, FIG. 1B) such that expression of the repressor-ATIC fusion placed under control of an isopropyl β-D-thiogalactoside (IPTG) inducible promoter. The fusion constructs showed IPTG dependent repression of the reporter genes on selective media, confirming the formation of a functional repressor. In order to improve selection conditions, a new RTHS strain was constructed by integrating the ATIC fusion onto the chromosome. The level of IPTG giving optimal repression was determined to be 50 μM by β-galactosidase assays.

The first SICLOPPS library transformed into the selection strain encoded a hexapeptide with five random residues and a cysteine nucleophile. Approximately 10⁷ transformants were plated onto histidine-free minimal media supplemented with arabinose, (inducer for SICLOPPS) 3-amino-1,2,4-triazole (3-AT, competitive inhibitor of HIS3 product) and kanamycin at a density of 10⁶ per plate (100×15 mm). The plates were incubated until colonies were readily visible (approximately one in 10⁵). A second library encoding an octapeptide with five random residues and an invariable SGW motif was also tested (not shown). Around 200 colonies were picked and screened for arabinose dependent growth advantage and IPTG dependent inhibition of growth to eliminate false positives. The expected phenotype was further confirmed by isolating and retransforming the selected SICLOPPS plasmids into the selection strain. The 14 remaining cyclic peptides were then ranked for activity by spotting serial dilutions of the corresponding cells onto selective media, allowing the conferred growth advantage to be compared at each dilution level.

To assess the in vivo target specificity of the selected cyclic peptides, a new RTHS strain containing a 434-repressor DNA-binding domain fusion with the Saccharomyces cerevisiae GCN4 leucine zipper (LZ) on its chromosome was constructed. The SICLOPPS plasmids of the active selectants were transformed into the LZ RTHS strain and ranked by drop spotting. ATIC specific cyclic peptide inhibitors were expected to be inactive in the LZ strain (identical to the ATIC RTHS strain except for the homodimer). Five of the 14 selectants incurred a growth advantage (arabinose dependent) on the LZ RTHS strain and were therefore discarded.

Materials.

All reagents were purchased from VWR Scientific or Sigma-Aldrich Fine Chemicals unless specified otherwise. Restriction and DNA-modifying enzymes were purchased from New England Biolabs. Oligonucleotides were purchased from Integrated DNA Technologies. Linear peptides were synthesized at the Hershey Macromolecular Core Facility of the Pennsylvania State University. Plasmid, PCR purification and gel extraction kits were purchased from Qiagen.

Recombinant DNA Techniques.

Escherichia coli cultures were maintained in LB broth. DNA manipulations were performed with E. coli DH5α-E (Invitrogen) cells. ATIC was cloned into pTHCP16 as a SalI/SacI fragment resulting in an in-frame fusion of the 434 repressor and ATIC coding sequences. Cloning and verification of DNA constructs was by standard techniques. Plasmids were transformed into E. coli by heat shock or electroporation. All DNA sequencing was performed at the Nucleic Acid Facility of the Pennsylvania State University.

Culture Media and Growth Conditions.

Antibiotics were provided at the following concentrations: ampicillin 100 μg/ml; chloroamphenicol 50 μg/ml; kanamycin 50 μg/ml; spectinomycin 50 μg/ml. For chromosomal markers, concentrations of antibiotics were reduced 2-fold. Minimal media A supplemented with 0.5% glycerol and 1 mM MgSO₄ was used for all genetic selections.

Genetic Selection.

SICLOPPS libraries were transformed into E. coli strains containing integrated reporter and repressor constructs. Transformants were washed with minimal media A and plated on minimal media A supplemented with 13 μM L-(+)-arabinose, 2.5 mM 3-amino-1,2,4-triazole, 25 μM kanamycin and 50 μM IPTG. After incubation at 37° C. for 3-4 days, surviving colonies were restreaked onto the same media with and without arabinose. Plasmids from selected strains whose growth depended on the presence of arabinose were retransformed into the original selection strain and checked for phenotype retention. The variable insert regions on SICLOPPS plasmids were PCR-amplified, and their DNA sequence determined.

Cyclic Peptide Synthesis.

Linear peptide 1a (RYFNVC, 10.0 mg, 12.5 μmol) (SEQ ID:19) was coupled onto chemically modified PEGA resin and cyclized as described in [Horswill et al. (2004) Proc. Natl. Acad. Sci. USA 101:15591] (6.3 mg, 8.0 μmol, 64%); m/z (MALDI) found 783.6 [C₃₆H₅₀N₁₀O₈S₁+H]⁺ requires 783.4.

Linear peptide 151 (WMFLNVSG, 10.0 mg, 10.5 μmol) (SEQ ID:20) was added to a solution of EDC (6 mg, 3 eq, 31.5 μmol) and HOAt (8.5 mg, 6 eq, 62.7 μmol) in DMF (15 ml). The mixture was agitated at room temperature for 24 hours. The solvent was removed in vacuo, the remaining residue was dissolved in 500 μl of DMF and added drop-wise to 10 ml of diethyl ether. The resulting solid was separated by centrifugation and purified as outlined below (7.2 mg, 7.7 μmol, 73%); m/z (MALDI) found 935.1 [C₄₅H₆₂N₁₀O₁₀S₁+H]⁺ requires 935.4. See FIG. 7D.

Crude cyclic peptides were subjected to reverse-phase chromatography (Partisil C-18 Magnum 9 {length 50 cm; particle size 10 μM} ODS-3 columns, Whatman) on a waters HPLC system by using a water/acetonitrile gradient with 0.1% trifluoroacetic acid. Mass Analysis was performed on a Mariner mass spectrometer (PerSeptive Biosystems, Framingham, Mass.).

Spectrophotometric Assays.

All assays were performed using a Varian Cary 100 Spectrometer. All reaction mixtures were 500 μl in volume and carried out in 1 cm pathlength quartz cuvettes at 25° C. The enzyme used in all of the inhibition studies was avian ATIC, fused to a N-terminal 6× histidine tag to facilitate purification. This fusion was constructed and verified by standard techniques. The peptides were dissolved in DMSO to a final concentration of 2.5 mM. The concentrations of DMSO used in the assay did not affect the activity of the enzyme.

AICAR Tfase Assay.

84 nM of ATIC, 50 μM of 10-f-THF and various quantities of inhibitor were mixed in the assay buffer (32.5 mM Tris-HCl, 25 mM KCl, pH 7.4). The mixture was incubated at 25° C. for 2 min before initiating the reaction by addition of 20 μM AICAR. The reaction was monitored by measuring the increase in absorbance due to formation of tetrahydrofolate at 298 nm.

IMPCH Assay.

To 100 μM of FAICAR in assay buffer (100 mM Tris-HCl, pH 7.4), 84 nM of ATIC was added. The reaction was monitored by monitoring the increase in absorbance due to the formation of IMP at 248 nm.

Progress Curve Analysis.

AICAR Tfase assays were conducted as outlined above. The inhibitors were assayed under two conditions, limiting the amount of each substrate. In one case 168 nM of ATIC, 100 μM of 10-f-THF and 20 μM of AICAR was used (limiting AICAR), and in the second case 168 nM of ATIC, 40 μM of 10-f-THF and 100 μM of AICAR was used (limiting 10-f-THF). The reactions were monitored as outlined above, for 50 minutes. Results of progress curve experiments were fit using the program DynaFit [P. Kuzmic, Anal Biochem (1996) 237:260] which is based in part upon KINSIM and FITSIM approaches [C. Frieden, Trends Biochem Sci (1993) 18:58]. The data was fitted to the standard inhibition models (non-competitive, uncompetitive, mixed and competitive) and a model in which the inhibitor binds a monomer of ATIC preventing dimerization.

### EXAMPLE 8

### Identification and Characterization of Cyclic AICAR Tfase Inhibitors Identified in Vivo

An inherent advantage of using genetically encoded libraries is the relative ease with which the structure of the active members can be determined (in contrast to deciphering synthetically derived libraries). Thus DNA sequencing of the variable inserts present on the selected SICLOPPS plasmids readily revealed the amino acid sequence of the ATIC specific cyclic peptide inhibitors (Table 5).

TABLE 5
Sequence of the selected cyclic peptides
in order of biological activity

| Activity | | |
|---|---|---|
| Rank | Name | Peptide sequence |
| 1 | c-1a (SEQ ID: 21) | R Y F N V C |
| 1 | c-151 (SEQ ID: 22) | M F L N V SGW |
| 2 | c-8 (SEQ ID: 23) | R I L Q L C |
| 2 | c-4 (SEQ ID: 24) | R F F I C C |
| 3 | c-6 (SEQ ID: 25) | T V L M F C |
| 3 | c-15 (SEQ ID: 26) | S M M V L C |
| 3 | c-5 (SEQ ID: 27) | R I L V L C |
| 3 | c-26 (SEQ ID: 28) | P V L L L C |
| 3 | c-25 (SEQ ID: 29) | M L L I V C |

There is considerable sequence homology in the genetically selected peptides. Overall, arginine is favored in position one; followed in position two by an aromatic amino acid (tyrosine or phenylalanine) in the more active, or an aliphatic amino acid (isoleucine, leucine or valine) in the less active cyclic peptides. The third random position is mainly occupied by leucine and phenylalanine. The three most active inhibitors contain an amino acid with an amide side chain (asparagine or glutamine) in position four. The fifth amino acid is mostly valine or leucine.

As the AICAR Tfase activity of ATIC is dependent on its dimerization, disruption of the homodimer can be monitered in vitro by AICAR Tfase assays. The two most active cyclic peptides (1a and 151) were chemically synthesized for in vitro characterization. Synthesis of cyclic peptide 1a involved immobilization of the corresponding linear sequence through its cysteine side chain on a modified amino polyethylene glycol acrylamide copolymer (PEGA) resin as a disulfide bond [Horswill et al. (2004) Proc. Natl. Acad. Sci. USA 101:15591]. The immobilized peptide was then cyclized, followed by cleavage off the PEGA resin (FIG. 7D). Linear peptide 151 was cyclized in N,N′-dimethylformamide (DMF) at high dilution to favor monomolecular cyclization.

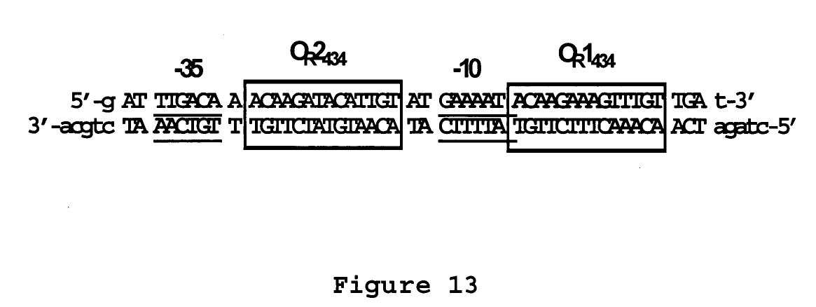

The cyclic peptides were purified by reverse phase chromatography. The chemical nature of the peptides was confirmed by comparison with biologically prepared samples (SICLOPPS) using reverse-phase HPLC and electrospray ionization mass spectrometry. Cyclic peptides c-1a and c-151 as well as their linear counterparts l-1a and l-151 were assayed against AICAR Tfase. The peptides were assumed to be competing with f-10-THF, which binds to ATIC and stabilizes its dimerization. From the measured k_{cat} of the enzyme (1.1 s⁻¹) and Kₘ of f-10-THF (33.9 μM), the competitive inhibition equation was used to determine the Kᵢ values (FIG. 13).

Peptide c-1a was found to have a Kᵢ of 17±4.2 μM whereas its linear counterpart l-1a has a Kᵢ of 142±22.5 μM. Inhibitor c-151 has a Kᵢ of 59±6.8 μM and again the linear peptide l-151 is less active with a Kᵢ of 173±28.4 μM. That both cyclic peptides were several times more potent than their linear counterparts confirms the superior activity of the genetically selected cyclic epitope and demonstrates the inherent entropic benefit of a constrained scaffold. The cyclic peptides were also assayed against IMPCH and showed no inhibitory effects. IMPCH activity is not dependent on enzyme dimerization, which suggests that the compounds act by inhibiting ATIC dimerization.

### EXAMPLE 9

### Verification of Homodimer Inhibition by in Vivo Selected Cyclic Peptides

The nature of the inhibition of the most active peptide, c-1a was verified by progress curve analysis [Kuzmic (1996) Anal. Biochem. 237:260; Stone et al. (1980) Biochemistry 19:620; Bauer et al. (1999) Biotechnol. Bioeng. 62:20; Frieden (1993) Trends Biochem. Sci. 18:58]. The progress curves were fitted to a model in which the inhibitor binds a single protomer of ATIC thereby preventing dimerization, as well as the standard inhibition models (non-competitive, uncompetitive, mixed and competitive) using DynaFit [Kuzmic (1996)]. The data for peptide c-1a best fitted the non-standard model (inhibition of enzyme dimerization) with respect to 10-f-THF, and the non-competitive inhibition model with respect to AICAR. This is consistent with both the ordered binding observed for the enzyme and stabilization of the catalytic dimer by 10-f-THF [Vergis et al. (2001) J. Biol. Chem. 276:7727; Bulock et al. (2002) J. Biol. Chem. 277:22168]. Furthermore the Kᵢ of c-1a obtained by this method (18±8.6 μM) closely matches that obtained assuming competitive inhibition with f-10-THF (17±4.2 μM). The collective kinetic data confirm that cyclic peptide 1a acts by inhibiting dimerization of ATIC (also indicated by the in vivo studies).

More potent inhibitors are evolved using second-generation SICLOPPS libraries (based on the selected sequences) and peptidomimetics [Andronati et al. (2004) Curr. Med. Chem. 11:1183].

In summary, we have demonstrated the genetic selection of cyclic peptide inhibitors of AICAR Tfase by combining the RTHS and SICLOPPS technologies. Nine cyclic peptides were selected from an intracellular library of 10⁸ members; these were confirmed to function by selective disruption of the ATIC homodimer in vivo and in vitro. These compounds represent a striking structural departure from traditional, antifolate-based inhibitors generally targeted against this enzyme [Cheong et al. (2004) J. Biol. Chem. 279:18034]. The reported methodology allowed rapid identification of small molecule inhibitors of protein-protein interactions, yielding a powerful and novel approach to drug discovery.

Each of the patents and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method for in vivo production and screening of the modulation of inter-macromolecule interaction comprising the steps of
a) providing a living cell that contains (i) a gene that directs expression of an exogenous gene product to be assayed for the ability to modulate inter-macromolecule interactions and (ii) inter-macromolecule interaction whose interaction can be monitored;
b) monitoring said inter-macromolecule interaction in said living cell; and
c) determining if said inter-macromolecule interaction is modulated in said living cell relative to another, otherwise similar living cell that lacks said gene product.

2. The method according to claim 1 wherein said cell is a prokaryote or a eukaryote.

3. The method according to claim 2 wherein said prokaryote is a bacterium.

4. The method according to claim 1 wherein said cell is a eukaryote.

5. The method according to claim 4 wherein eukaryote is a yeast, animal, or plant cell.

6. The method according to claim 1 wherein said gene product is a small molecule, a macrolide or a nucleic acid.

7. The method according to claim 6 wherein said small molecule is a peptide having a sequence of about 4 to about 150 residues.

8. The method according to claim 1 wherein said gene product is a macrolide.

9. The method according to claim 1 wherein said macrolide is rapamycin.

10. The method according to claim 1 wherein said inter-macromolecule interaction is a protein-protein interaction.

11. The method according to claim 1 wherein said exogenous gene comprises a library of genes.

12. The method according to claim 6 wherein said exogenous gene comprises a library of genes.

13. The method according to claim 1 wherein said monitoring comprises observation of cell growth, enzyme activity or both cell growth and enzyme activity.

14. A living cell that contains i) an exogenous gene that directs expression of a gene product to be assayed for the ability to modulate inter-macromolecule interactions and (ii) inter-macromolecule interaction whose interaction can be monitored by comparing said living cell to an other, similar living cell lacking said gene product.

15. The living cell according to claim 14 wherein said gene comprises a library of genes.

16. The living cell according to claim 14 wherein said monitoring comprises observation of cell growth, enzyme activity or both cell growth and enzyme activity in said living cell.


SEQUENCE LISTING (29 sequences)

SEQ ID NO: 1 - DNA, 58 bp, Artificial sequence
  Hybrid promoter containing two repressor binding sites
ctagatatttaagatttcttgtattttcatttaagatatcttgtttgtcaaatctgca

SEQ ID NO: 2 - DNA, 48 bp, Artificial sequence
  Promoter containing two wild-type 434 repressor binding sites
acaagatttcttgtattttcacaagatatcttgtttgtcaaatctgca

SEQ ID NO: 3 - protein, 5 aa, Artificial sequence
  ribonucleotide reductase inhibitor peptide
VKFWF

SEQ ID NO: 4 - protein, 5 aa, Artificial sequence
  Ribonucleotide reductase inhibitor peptide
RYYNV

SEQ ID NO: 5 - protein, 5 aa, Artificial sequence
  Ribonucleotide reductase inhibitor peptide
YTWSY

SEQ ID NO: 6 - protein, 5 aa, Artificial sequence
  Ribonucleotide reductase inhibitor peptide
IPLLY

SEQ ID NO: 7 - protein, 5 aa, Artificial sequence
  Ribonucleotide reductase inhibitor peptide
GVRFF

SEQ ID NO: 8 - protein, 5 aa, Artificial sequence
  Ribonucleotide reductase inhibitor peptide
LNYLW

SEQ ID NO: 9 - protein, 5 aa, Artificial sequence
  Ribonucleotide reductase inhibitor peptide
HRYVF

SEQ ID NO: 10 - protein, 5 aa, Artificial sequence
  Ribonucleotide reductase inhibitor peptide
KISLF

SEQ ID NO: 11 - protein, 5 aa, Artificial sequence
  Ribonucleotide reductase inhibitor peptide
VLYSW

SEQ ID NO: 12 - protein, 6 aa, Artificial sequence
  Insert for target cyclic peptide 1
CXXXXX

SEQ ID NO: 13 - protein, 8 aa, Artificial sequence
  Insert for cyclic target peptide 2
SGWXXXXX

SEQ ID NO: 14 - DNA, 59 bp, Artificial sequence
  Promoter containing two wild-type 434 repressor binding sites
ctagatcaacaaaactttcttgtattttcatacaatgtatcttgtttgtcaaatctgca

SEQ ID NO: 15 - protein, 9 aa, Artificial sequence
  Expressed product from oligo, 8 R2 subunit C-terminal
MSFTLDADF

SEQ ID NO: 16 - protein, 9 aa, Artificial sequence
  Expressed product, scrambled peptide control
MDTAFSFLD

SEQ ID NO: 17 - protein, 7 aa, Artificial sequence
  Expressed product for methionine plus hexapeptide control peptide
MTVSYEL

SEQ ID NO: 18 - protein, 7 aa, Artificial sequence
  Expression product methionine plus control peptide
MDSATYV

SEQ ID NO: 19 - protein, 6 aa, Artificial sequence
  ATIC dimerization inhibitor, linear peptide 1a
RYFNVC

SEQ ID NO: 20 - protein, 8 aa, Artificial sequence
  ATIC dimerization inhibitor, linear peptide 151
WMFLNVSG

SEQ ID NO: 21 - protein, 6 aa, Artificial sequence
  ATIC dimerization inhibitor 1a circular
RYFNVC

SEQ ID NO: 22 - protein, 8 aa, Artificial sequence
  ATIC dimerization inhibitor 151b
MFLNVSGW

SEQ ID NO: 23 - protein, 6 aa, Artificial sequence
  ATIC dimerization ihnibitor 8
RILQLC

SEQ ID NO: 24 - protein, 6 aa, Artificial sequence
  ATIC dimerization inhibitor 4
RFFICC

SEQ ID NO: 25 - protein, 6 aa, Artificial sequence
  ATIC dimerization inhibitor 6
TVLMFC

SEQ ID NO: 26 - protein, 6 aa, Artificial sequence
  ATIC dimerization inhibitor
SMMVLC

SEQ ID NO: 27 - protein, 6 aa, Artificial sequence
  ATIC dimerization inhibitor 5
RILVLC

SEQ ID NO: 28 - protein, 6 aa, Artificial sequence
  ATIC dimerization inibitor 26
PVLLLC

SEQ ID NO: 29 - protein, 6 aa, Artificial sequence
  ATIC dimerization inhibitor 25
MLLIVC